(19) United States
(12) Patent Application Publication
(10) Pub. No.: US 2021/0322412 A1
(43) Pub. Date: Oct. 21, 2021

(54) METHODS AND COMPOSITIONS FOR TREATING A CORONAVIRUS INFECTION

(71) Applicant: Centrexion Therapeutics Corporation, Boston, MA (US)

(72) Inventors: James N. Campbell, Baltimore, MD (US); Randall Stevens, Rockport, MA (US)

(21) Appl. No.: 17/234,867

(22) Filed: Apr. 20, 2021

Related U.S. Application Data
(60) Provisional application No. 63/012,519, filed on Apr. 20, 2020.

(51) Int. Cl. A61K 31/506

(52) U.S. Cl. CPC A61K 31/506

(57) ABSTRACT

The invention provides methods, compositions, and kits for using substituted (4-(amino)piperidin-1-yl)(6-(((tetrahydro-2H-pyran-2-yl)methyl)amino)pyrimidin-4-yl)methanone compounds to treat a coronavirus infection and related medical conditions.

### CROSS REFERENCE TO RELATED APPLICATIONS

This application claims the benefit of and priority to U.S. Provisional Patent Application Ser. No. 63/012,519, filed Apr. 20, 2020, the contents of which are hereby incorporated by reference.

### FIELD OF THE INVENTION

The invention provides methods, compositions, and kits for using substituted (4-(amino)piperidin-1-yl)(6-(((tetrahydro-2H-pyran-2-yl)methyl)amino)pyrimidin-4-yl)methanone compounds to treat a coronavirus infection and related medical conditions.

### BACKGROUND

Coronavirus infections have been responsible for multiple deadly outbreaks. The severe acute respiratory syndrome (SARS) outbreak in years 2002-2004 resulted in over 700 fatalities globally. The Middle East respiratory syndrome (MERS) outbreak in years 2012-2015 also resulted in many fatalities globally. The severe acute respiratory syndrome coronavirus 2 (SARS-CoV-2) outbreak that started in year 2019 has resulted in the death of a large number of people in many different countries. Existing treatments have proven inadequate leading to the need for new treatments for coronavirus infections.

The present invention addresses this need and provides other related advantages.

### SUMMARY

The invention provides methods, compositions, and kits for using substituted (4-(amino)piperidin-1-yl)(6-(((tetrahydro-2H-pyran-2-yl)methyl)amino)pyrimidin-4-yl)methanone compounds to treat a coronavirus infection and related medical conditions. The coronavirus infection may be, for example, an infection by SARS-CoV-2. The compound may be a compound of Formula I, or a pharmaceutically acceptable salt thereof, for example, (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate. The methods, compositions, and medical kits of the present disclosure provide particular benefits to patients suffering from pulmonary inflammation related to a coronavirus infection.

Accordingly, one aspect of the invention provides a method of treating a coronavirus infection. The method comprises administering to a patient in need thereof a therapeutically effective amount of a compound of Formula I in order to treat the coronavirus infection, wherein Formula I is represented by:

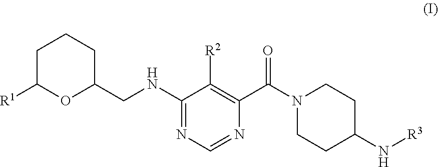

or a pharmaceutically acceptable salt thereof, where the variables are as defined in the detailed description. Further description of additional collections of substituted (4-(amino)piperidin-1-yl)(6-(((tetrahydro-2H-pyran-2-yl)methyl)amino)pyrimidin-4-yl)methanone compounds are described in the detailed description. The compounds may be part of a pharmaceutical composition comprising a pharmaceutically acceptable carrier.

Another aspect of the invention provides a method of reducing inflammation in a patient suffering from a coronavirus infection. The method comprises administering to a patient in need thereof a therapeutically effective amount of a compound of Formula I, or a pharmaceutically acceptable salt thereof, in order to reduce inflammation, as further described in the detailed description.

Another aspect of the invention provides a method of reducing the impact of a pro-inflammatory cytokine in a patient suffering from a coronavirus infection. The method comprises administering to a patient in need thereof a therapeutically effective amount of a compound of Formula I, or a pharmaceutically acceptable salt thereof, in order to reduce the impact of a pro-inflammatory cytokine, as further described in the detailed description. The reduction in impact of the pro-inflammatory cytokine may be, for example, a reduction in inflammation in the patient, such as a reduction in pulmonary inflammation in the patient.

Another aspect of the invention provides a method for the prophylaxis of lung injury in a patient suffering from a coronavirus infection. The method comprises administering to a patient in need thereof a therapeutically effective amount of a compound of Formula I, or a pharmaceutically acceptable salt thereof, as further described in the detailed description. The lung injury may be, for example, injury due to inflammation.

Another aspect of the invention provides a method of treating a coronavirus infection. The method comprises administering to a patient in need thereof a therapeutically effective amount of a compound having inhibitory activity towards both the chemokine receptor 2 and the chemokine receptor 5, in order to treat the coronavirus infection. In certain embodiments, the compound has an IC₅₀ less than 150 nM towards chemokine receptor 2. In certain embodiments, the compound has an IC₅₀ less than 150 nM towards chemokine receptor 5

The foregoing aspects of the invention are described in more detail, along with additional embodiments, in the detailed description below.

### BRIEF DESCRIPTION OF THE DRAWINGS

FIG. 1 shows the X-ray powder diffractogram of the amorphous base of the compound (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone.

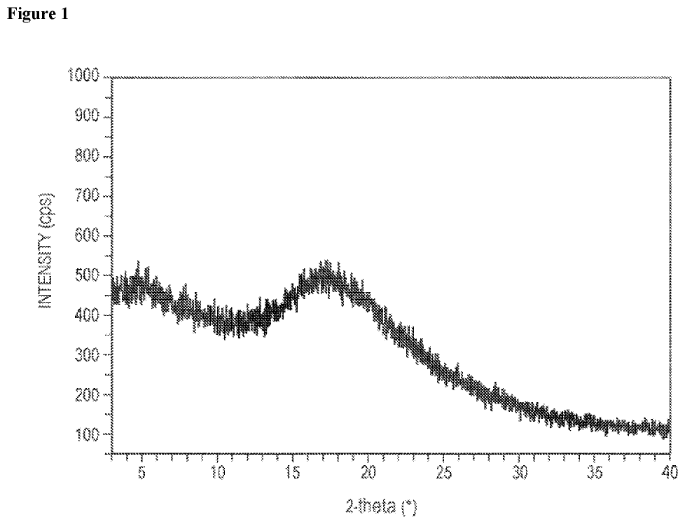

FIG. 2 shows the X-ray powder diffractogram of (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate.

FIG. 3 shows the thermoanalysis and determination of the melting point (DSC/TG) of (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate.

FIG. 4 shows the sorption isotherms of (4-((3R,4R)-3-methoxytetrahydro-pyran-4-ylamino)piperidin-1-yl)(5-methyl-6-((2R,6S)-6-(p-tolyl)tetrahydro-2H-pyran-2-yl)methylamino)pyrimidin-4-yl)methanone citrate.

### DETAILED DESCRIPTION OF THE INVENTION

The invention provides methods, compositions, and kits for using substituted (4-(amino)piperidin-1-yl)(6-(((tetrahydro-2H-pyran-2-yl)methyl)amino)pyrimidin-4-yl)methanone compounds to treat a coronavirus infection and related medical conditions. The coronavirus infection may be, for example, an infection by SARS-CoV-2. The compound may be a compound of Formula I, or a pharmaceutically acceptable salt thereof, for example, (4-((3R,4R)-3-methoxytetrahydro-pyran-4-ylamino)piperidin-1-yl)(5-methyl-6-((2R,6S)-6-(p-tolyl)tetrahydro-2H-pyran-2-yl)methylamino)pyrimidin-4-yl)methanone citrate. The methods, compositions, and medical kits of the present disclosure provide particular benefits to patients suffering from pulmonary inflammation related to a coronavirus infection.

The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The terms “a,” “an” and “the” as used herein mean “one or more” and include the plural unless the context is inappropriate.

The term “about” means within 10% of the stated value. In certain embodiments, the value may be within 8%, 6%, 4%, 2%, or 1% of the stated value.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “halocycloalkyl” refers to a cycloalkyl group that is substituted with at least one halogen.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-7 membered heteroalkyl refers to a heteroalkyl group having from 3 to 8 atoms selected from the group consisting of carbon and a heteroatom.

The term “aralkyl” refers to an alkyl group substituted with an aryl group. Exemplary aralkyl groups include

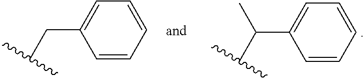

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “cycloalkenyl” refers to a monovalent unsaturated cyclic, bicyclic, or bridged (e.g., adamantyl) carbocyclic hydrocarbon containg at least one C—C double bond. In certain embodiments, the cycloalkenyl contains 5-10, 5-8, or 5-6 carbons, referred to herein, e.g., as “C₅-C₆ cycloalkenyl”. Exemplary cycloalkenyl groups include cyclohexenyl and cyclopentenyl.

The term “carbocyclyl” refers to a mono-radical of a saturated, partially unsaturated, or aromatic carbocyclic ring (e.g., a monocyclic, bicyclic, bridged (e.g., adamantyl), or spirocyclic ring). In certain embodiments, the carbocyclyl contains 3-10, 4-8, or 5-6 carbons, referred to herein, e.g., as “C₅-C₆ carbocyclyl”.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “heteroaryl” is art-recognized and refers to aromatic groups that include at least one ring heteroatom. In certain instances, a heteroaryl group contains 1, 2, 3, or 4 ring heteroatoms (e.g., O, N, and S). Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic aromatic ring systems having two or more rings in which two or more ring atoms are common to two adjoining rings (the rings are “fused rings”) wherein all of the fused rings are heteroaromatic, e.g., in a naphthyridinyl group. In certain embodiments, the heteroaryl is a 5-6 membered monocylic ring or a 9-10 membered bicyclic ring.

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

As used herein, the terms “heterocyclic” and “heterocyclyl” refer to a saturated, partially unsaturated, or aromatic ring (e.g., a monocyclic, bicyclic ring, bridged, or spirocyclic ring) containing one or more heteroatoms (e.g., 1, 2, 3, or 4 heteroatoms, such as where the heteroatom is selected from oxygen, nitrogen, and sulfur). The heteroatoms can be the same or different from each other. Examples of heteratoms include, but are not limited to nitrogen, oxygen and sulfur. Unless specified otherwise, the heterocyclic ring is optionally substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. Aromatic and nonaromatic heterocyclic rings are well-known in the art. Some nonlimiting examples of aromatic heterocyclic rings include, but are not limited to, pyridine, pyrimidine, indole, purine, quinoline and isoquinoline. Nonlimiting examples of nonaromatic heterocyclic compounds include, but are not limited to, piperidine, piperazine, morpholine, pyrrolidine and pyrazolidine. Examples of oxygen containing heterocyclic rings include, but are not limited to, furan, oxirane, 2H-pyran, 4H-pyran, 2H-chromene, benzofuran, and 2,3-dihydrobenzo[b][1,4]dioxine. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. In certain embodiments, the heterocyclyl group is a 3-7 membered ring that, unless specified otherwise, is substituted or unsubstituted. In certain embodiments, the heterocyclyl group is a 3-7 membered ring that contains 1, 2, or 3 ring heteroatoms selected from oxygen, sulfur, and nitrogen.

The term “heterocycloalkyl” refers to a saturated heterocyclyl group having, for example, 3-7 ring atoms selected from carbon and heteroatoms (e.g., O, N, or S).

The terms “amine” and “amino” are art-recognized and refer to both unsubstituted and substituted amines, e.g., a moiety that may be represented by the general formulas:

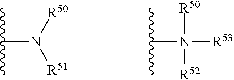

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and —O—(CH₂)ₘ—R⁶¹, where m and R⁶¹ are described above.

The term “oxo” is art-recognized and refers to a “═O” substituent. For example, a cyclopentane susbsituted with an oxo group is cyclopentanone.

The symbol “ [symbol] ” indicates a point of attachment.

When depicted at the end of a chemical bond, the symbol “*” indicates a point of attachment.

The term “substituted” means that one or more hydrogens on the atoms of the designated group are replaced with a selection from the indicated group, provided that the atoms' normal valencies under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for therapeutic use in vivo or ex vivo.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “therapeutically effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for treating a coronavirus infection, reducing inflammation in a patient suffering from a coronavirus infection, reducing the impact of a pro-inflammatory cytokine in a patient suffering from a coronavirus infection, and prophylaxis of lung injury in a patient suffering from a coronavirus infection. The methods involve administering to a patient a substituted (4-(amino)piperidin-1-yl)(6-(((tetrahydro-2H-pyran-2-yl)methyl)amino)pyrimidin-4-yl)methanone compound, such as a compound of Formula I, or a pharmaceutically acceptable salt thereof, for example, (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate. The coronavirus infection may be, for example, an infection by SARS-CoV-2.

One common feature of a coronavirus infection is increased incidence of inflammation in the patient, particularly increased incidence of inflammation in pulmonary tissue. The increased incidence of inflammation is believed to be attributed to an increased concentration of pro-inflammatory cytokines in the patient suffering from the coronavirus infection. The substituted (4-(amino)piperidin-1-yl)(6-(((tetrahydro-2H-pyran-2-yl)methyl)amino)pyrimidin-4-yl)methanone compounds described herein, such as a compound of Formula I, have inhibitory activity towards both chemokine receptor 2 (CCR2) and chemokine receptor 5 (CCR5). Inhibitory activity towards CCR2 and CCR5 is believed to provide a therapeutic benefit in treating a coronavirus infection and reducing and/or preventing inflammation in the patient, in part because CCR2 and CCR5 contribute to inflammatory response in a patient. CCR2 activity, for example, impacts recruitment of monocytes/macrophages to an inflammatory site. CCR5 activity, for example, is involved in directing leukocytes to a site of inflammation. Accordingly, the methods, compositions, and medical kits of the present disclosure provide particular benefits to patients suffering from a coronavirus infection.

Various aspects and embodiments of the therapeutic methods are described in the sections below. The sections are arranged for convenience and information in one section is not to be limited to that section, but may be applied to methods in other sections.

### A. Methods for Treating a Coronavirus Infection

One aspect of the invention provides a method of treating a coronavirus infection. The method comprises administering to a patient in need thereof a therapeutically effective amount of a compound of Formula I in order to treat the coronavirus infection, wherein Formula I is represented by:

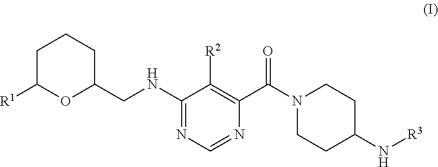

or a pharmaceutically acceptable salt thereof, wherein:

R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;

R² is C₁₋₆ alkyl or C₁₋₆ haloalkyl; and

R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

In certain embodiments, the patient presents with inflammation due to the coronavirus infection.

B. Methods for Reducing Inflammation in a Patient Suffering from a Coronavirus Infection

Another aspect of the invention provides methods of reducing inflammation in a patient suffering from a coronavirus infection. The method comprises administering to a patient in need thereof an effective amount of a compound of Formula I in order to reduce inflammation, wherein Formula I is represented by:

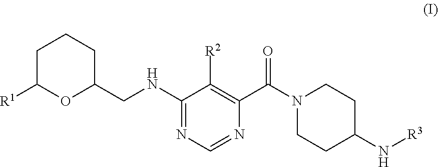

or a pharmaceutically acceptable salt thereof, wherein:

R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;

R² is C₁₋₆ alkyl or C₁₋₆ haloalkyl; and

R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

Results achieved by the therapeutic method can be characterized according to, for example, the reduction of inflammation. For example, in certain embodiments, at least a 25% reduction in the incidence of inflammation is achieved after 1 week from first administering the compound of Formula Ito the patient. In certain embodiments, at least a 50% reduction in the incidence of inflammation is achieved after 1 week from first administering the compound of Formula Ito the patient. In certain embodiments, at least a 75% reduction in the incidence of inflammation is achieved after 1 week from first administering the compound of Formula Ito the patient. In certain embodiments, at least a 90% reduction in the incidence of inflammation is achieved after 1 week from first administering the compound of Formula I to the patient.

The reduction in inflammation may be further characterized according to, for example, a reduction in the volume of pulmonary tissue presenting with inflammation. In certain embodiments, there is at least a 25% reduction in the volume of pulmonary tissue presenting with inflammation after 1 week from first administering the compound of Formula Ito the patient. In certain embodiments, there is at least a 50% reduction in the volume of pulmonary tissue presenting with inflammation after 1 week from first administering the compound of Formula Ito the patient. In certain embodiments, there is at least a 75% reduction in the volume of pulmonary tissue presenting with inflammation after 1 week from first administering the compound of Formula Ito the patient. In certain embodiments, there is at least a 90% reduction in the volume of pulmonary tissue presenting with inflammation after 1 week from first administering the compound of Formula Ito the patient.

Inflammation may be measured according to a variety of methods known to one of skill in the art. For example, in certain embodiments, the inflammation is measured according to the amount of pro-inflammatory cytokines in the patient. In certain embodiments, the inflammation is measured according to the amount in the patient of one or more of IL-1, IL-2, IL-6, IL-7, granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1α), and tumor necrosis factor-α (TNF-α). In certain embodiments, the inflammation is measured according to the amount in the patient of one or more of IL-1, IL-2, IL-6, and IL-7.

In certain embodiments, the inflammation is measured in a tissue sample of the patient. In certain embodiments, the inflammation is measured in a pulmonary tissue sample of the patient. In certain embodiments, the inflammation is measured by measuring the amount of an inflammation biomarker (e.g., pro-inflammatory cytokine) in blood plasma of the patient.

C. Methods for Reducing the Impact of a Pro-Inflammatory Cytokine in a Patient Suffering from a Coronavirus Infection

Another aspect of the invention provides methods of reducing the impact of a pro-inflammatory cytokine in a patient suffering from a coronavirus infection. The method comprises administering to a patient in need thereof an effective amount of a compound of Formula I in order to reduce the impact of a pro-inflammatory cytokine in a patient suffering from a coronavirus infection, wherein Formula I is represented by:

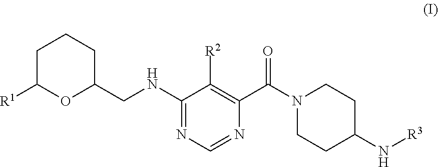

or a pharmaceutically acceptable salt thereof, wherein:

R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;

R² is C₁₋₆ alkyl or C₁₋₆ haloalkyl; and

R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

In certain embodiments, the impact of pro-inflammatory cytokine comprises increased inflammation. In certain embodiments, the impact of pro-inflammatory cytokine comprises respiratory failure, septic shock, and/or multi-organ failure.

The method can be characterized according to, for example, the identity of the pro-inflammatory cytokine. For example, in certain embodiments, the pro-inflammatory cytokine is IL-1, IL-2, IL-6, or IL-7. In certain embodiments, the pro-inflammatory cytokine is IL-1. In certain embodiments, the pro-inflammatory cytokine is IL-2. In certain embodiments, the pro-inflammatory cytokine is IL-6. In certain embodiments, the pro-inflammatory cytokine is IL-7.

In certain embodiments, the pro-inflammatory cytokine is IL-2, IL-7, IL-6, granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1α), or tumor necrosis factor-α (TNF-α). In certain embodiments, the pro-inflammatory cytokine is granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1α), or tumor necrosis factor-α (TNF-α).

The method can be characterized according to, for example, the concentration of the pro-inflammatory cytokine in the patient. For example, in certain embodiments, the patient has a concentration of the pro-inflammatory cytokine that is greater than the average concentration in a healthy patient. In certain embodiments, the patient has a concentration of the pro-inflammatory cytokine that is at least 5%, 10%, 20%, 30%, 40%, 50%, 60%, 70%, 80%, 90% or 100% greater than the average concentration in a healthy patient. In certain embodiments, the concentration of the pro-inflammatory cytokine is measured in the blood plasma of the patient. In certain embodiments, the concentration of the pro-inflammatory cytokine is measured in a tissue sample of the patient. In certain embodiments, the concentration of the pro-inflammatory cytokine is measured in a pulmonary tissue sample of the patient.

In certain embodiments, the patient has a blood plasma concentration of one or more of IL-2, IL-7, IL-6, granulocyte-macrophage colony-stimulating factor (GM-CSF), interferon-γ inducible protein 10 (IP-10), monocyte chemoattractant protein 1 (MCP-1), RANTES, macrophage inflammatory protein 1-α (MIP-1α), or tumor necrosis factor-α (TNF-α) that is greater than the average blood plasma concentration in a healthy patient. RANTES (which stands for: regulated on activation, normal T cell expressed and secreted) is the endogenous ligand for CCRS. In certain embodiments, the blood plasma concentration of one or more of IL-2, IL-7, IL-6, granulocyte-macrophage colony-stimulating factor (GM-CSF), interferon-γ inducible protein 10 (IP-10), monocyte cheurmoattractant protein 1 (MCP-1), macrophage inflammatory protein 1-α (MIP-1α), or tumor necrosis factor-α (TNF-α) is at least 5%, 10%, 20%, 30%, 40%, 50%, 60%, 70%, 80%, 90% or 100% greater than the average blood plasma concentration in a healthy patient.

In certain embodiments, the concentration of activated macrophages in the patient is greater than the corresponding tissue average concentration of activated macrophages in a healthy patient. In certain embodiments, the concentration of activated macrophages in a tissue in the patient is at least 5%, 10%, 20%, 30%, 40%, 50%, 60%, 70%, 80%, 90% or 100% greater than the average concentration of activated macrophages in the corresponding tissue of a healthy patient. In certain embodiments, said tissue is the patient's lung, kidney, or liver.

D. Methods for the Prophylaxis of Lung Injury in a Patient Suffering from a Coronavirus Infection

Another aspect of the invention provides methods for the prophylaxis of lung injury in a patient suffering from a coronavirus infection. The method comprises administering to a patient in need thereof a therapeutically effective amount of a compound of Formula I:

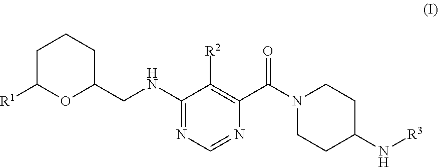

or a pharmaceutically acceptable salt thereof, wherein:

R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;

R² is C₁₋₆ alkyl or C₁₋₆ haloalkyl; and

R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

### E. Methods for Treating a Coronavirus Infection Using a Compound With Dual CCR2 and CCR5 Inhibitory Activity

Another aspect of the invention provides a method of treating a coronavirus infection. The method comprises administering to a patient in need thereof a therapeutically effective amount of a compound having inhibitory activity towards both the chemokine receptor 2 and the chemokine receptor 5, in order to treat the coronavirus infection.

The method may be further characterized according to, for example, the identity of the compound having inhibitory activity towards both the chemokine receptor 2 and the chemokine receptor 5. In certain embodiments, the compound has an IC₅₀ less than 0.5 μM towards chemokine receptor 2. In certain embodiments, the compound has an IC₅₀ less than 150 nM towards chemokine receptor 2. In certain embodiments, the compound has an IC₅₀ less than 0.5 μM towards chemokine receptor 5. In certain embodiments, the compound has an IC₅₀ less than 150 nM towards chemokine receptor 5. In certain embodiments, the compound has a molecular weight less than 900 g/mol.

F. Methods for Reducing Inflammation in a Patient Suffering from a Coronavirus Infection Using a Compound With Dual CCR2 and CCR5 Inhibitory Activity

Another aspect of the invention provides methods of reducing inflammation in a patient suffering from a coronavirus infection. The method comprises administering to a patient in need thereof an effective amount of a compound having inhibitory activity towards both the chemokine receptor 2 and the chemokine receptor 5, in order to reduce inflammation.

Results achieved by the method can be characterized according to, for example, the reduction of inflammation achieved by the method. For example, in certain embodiments, at least a 25% reduction in the incidence of inflammation is achieved after 1 week from first administering the compound to the patient. In certain embodiments, at least a 50% reduction in the incidence of inflammation is achieved after 1 week from first administering the compound to the patient. In certain embodiments, at least a 75% reduction in the incidence of inflammation is achieved after 1 week from first administering the compound to the patient. In certain embodiments, at least a 90% reduction in the incidence of inflammation is achieved after 1 week from first administering the compound to the patient.

The reduction in inflammation may be further characterized according to, for example, a reduction in the volume of pulmonary tissue presenting with inflammation. In certain embodiments, at least a 25% reduction in the volume of pulmonary tissue presenting with inflammation after 1 week from first administering the compound to the patient. In certain embodiments, at least a 50% reduction in the volume of pulmonary tissue presenting with inflammation after 1 week from first administering the compound to the patient. In certain embodiments, at least a 75% reduction in the volume of pulmonary tissue presenting with inflammation after 1 week from first administering the compound to the patient. In certain embodiments, at least a 90% reduction in the volume of pulmonary tissue presenting with inflammation after 1 week from first administering the compound to the patient.

The method may be further characterized according to, for example, the identity of the compound having inhibitory activity towards both the chemokine receptor 2 and the chemokine receptor 5. In certain embodiments, the compound has an IC₅₀ less than 0.5 μM towards chemokine receptor 2. In certain embodiments, the compound has an IC₅₀ less than 150 nM towards chemokine receptor 2. In certain embodiments, the compound has an IC₅₀ less than 0.5 μM towards chemokine receptor 5. In certain embodiments, the compound has an IC₅₀ less than 150 nM towards chemokine receptor 5. In certain embodiments, the compound has a molecular weight less than 900 g/mol.

G. Methods for Reducing the Impact of a Pro-Inflammatory Cytokine in a Patient Suffering from a Coronavirus Infection Using a Compound With Dual CCR2 and CCR5 Inhibitory Activity

Another aspect of the invention provides methods of reducing the impact of a pro-inflammatory cytokine in a patient suffering from a coronavirus infection. The method comprises administering to a patient in need thereof an effective amount of a compound having inhibitory activity towards both the chemokine receptor 2 and the chemokine receptor 5, in order to reduce the impact of a pro-inflammatory cytokine in a patient suffering from a coronavirus infection.

In certain embodiments, the impact of pro-inflammatory cytokine comprises increased inflammation. In certain embodiments, the impact of pro-inflammatory cytokine comprises respiratory failure, septic shock, and/or multi-organ failure.

The method can be characterized according to, for example, the identity of the pro-inflammatory cytokine. For example, in certain embodiments, the pro-inflammatory cytokine is IL-1, IL-2, IL-6, or IL-7. In certain embodiments, the pro-inflammatory cytokine is IL-1. In certain embodiments, the pro-inflammatory cytokine is IL-2. In certain embodiments, the pro-inflammatory cytokine is IL-6. In certain embodiments, the pro-inflammatory cytokine is IL-7.

In certain embodiments, the pro-inflammatory cytokine is IL-2, IL-7, IL-6, granulocyte-macrophage colony-stimulating factor (GM-CSF), interferon-α inducible protein 10 (IP-10), monocyte chemoattractant protein 1 (MCP-1), macrophage inflammatory protein 1-α (MIP-1α), or tumor necrosis factor-α (TNF-α). In certain embodiments, the pro-inflammatory cytokine is granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1α), or tumor necrosis factor-α (TNF-α).

The method can be characterized according to, for example, the concentration of the pro-inflammatory cytokine in the patient. For example, in certain embodiments, the patient has a concentration of the pro-inflammatory cytokine that is greater than the average concentration in a healthy patient. In certain embodiments, the patient has a concentration of the pro-inflammatory cytokine that is at least 5%, 10%, 20%, 30%, 40%, 50%, 60%, 70%, 80%, 90% or 100% greater than the average concentration in a healthy patient. In certain embodiments, the concentration of the pro-inflammatory cytokine is measured in the blood plasma of the patient. In certain embodiments, the concentration of the pro-inflammatory cytokine is measured in a tissue sample of the patient. In certain embodiments, the concentration of the pro-inflammatory cytokine is measured in a pulmonary tissue sample of the patient.

In certain embodiments, the patient has a blood plasma concentration of one or more of IL-2, IL-7, IL-6, granulocyte-macrophage colony-stimulating factor (GM-CSF), interferon-γ inducible protein 10 (IP-10), monocyte chemoattractant protein 1 (MCP-1), RANTES, macrophage inflammatory protein 1-α (MIP-1α), or tumor necrosis factor-α (TNF-α) that is greater than the average blood plasma concentration in a healthy patient. In certain embodiments, the blood plasma concentration of one or more of IL-2, IL-7, IL-6, granulocyte-macrophage colony-stimulating factor (GM-CSF), interferon-y inducible protein 10 (IP-10), monocyte chemoattractant protein 1 (MCP-1), macrophage inflammatory protein 1-α (MIP-1α), or tumor necrosis factor-α (TNF-α) is at least 5%, 10%, 20%, 30%, 40%, 50%, 60%, 70%, 80%, 90% or 100% greater than the average blood plasma concentration in a healthy patient.

In certain embodiments, the concentration of activated macrophages in the patient is greater than the corresponding tissue average concentration of activated macrophages in a healthy patient. In certain embodiments, the concentration of activated macrophages in a tissue in the patient is at least 5%, 10%, 20%, 30%, 40%, 50%, 60%, 70%, 80%, 90% or 100% greater than the average concentration of activated macrophages in the corresponding tissue of a healthy patient. In certain embodiments, said tissue is the patient's lung, kidney, or liver.

The method may be further characterized according to, for example, the identity of the compound having inhibitory activity towards both the chemokine receptor 2 and the chemokine receptor 5. In certain embodiments, the compound has an IC₅₀ less than 0.5 μM towards chemokine receptor 2. In certain embodiments, the compound has an IC₅₀ less than 150 nM towards chemokine receptor 2. In certain embodiments, the compound has an IC₅₀ less than 0.5 μM towards chemokine receptor 5. In certain embodiments, the compound has an IC₅₀ less than 150 nM towards chemokine receptor 5. In certain embodiments, the compound has a molecular weight less than 900 g/mol.

H. Methods for the Prophylaxis of Lung Injury in a Patient Suffering from a Coronavirus Infection Using a Compound With Dual CCR2 and CCR5 Inhibitory Activity

Another aspect of the invention provides methods for the prophylaxis of lung injury in a patient suffering from a coronavirus infection. The method comprises administering to a patient in need thereof a therapeutically effective amount of a compound having inhibitory activity towards both the chemokine receptor 2 and the chemokine receptor 5.

The method may be further characterized according to, for example, the identity of the compound having inhibitory activity towards both the chemokine receptor 2 and the chemokine receptor 5. In certain embodiments, the compound has an IC₅₀ less than 0.5 μM towards chemokine receptor 2. In certain embodiments, the compound has an IC₅₀ less than 150 nM towards chemokine receptor 2. In certain embodiments, the compound has an IC₅₀ less than 0.5 μM towards chemokine receptor 5. In certain embodiments, the compound has an IC₅₀ less than 150 nM towards chemokine receptor 5. In certain embodiments, the compound has a molecular weight less than 900 g/mol.

I. Methods for Reducing Activity of Inflammatory Macrophages in a Patient Suffering from a Coronavirus Infection Using a Compound With Dual CCR2 and CCR5 Inhibitory Activity

Another aspect of the invention provides methods of reducing activity of inflammatory macrophages in a patient suffering from a coronavirus infection. The method comprises administering to a patient in need thereof an effective amount of a compound having inhibitory activity towards both the chemokine receptor 2 and the chemokine receptor 5, in order to reduce activity of inflammatory macrophages.

In certain embodiments, reducing the activity of inflammatory macrophages comprises reducing recruitment of inflammatory macrophages. In certain embodiments, reducing the activity of inflammatory macrophages comprises reducing recruitment of inflammatory macrophages to a site of coronavirus infection. In certain embodiments, reducing the activity of inflammatory macrophages comprises reducing recruitment of inflammatory macrophages to lung, kidney, liver, heart, brain or other vital organs in the patient. In certain embodiments, reducing the activity of inflammatory macrophages comprises reducing recruitment of inflammatory macrophages to the lung, kidney, liver, heart, or brain.

In certain embodiments, reducing the activity of inflammatory macrophages comprises reducing activation of inflammatory macrophages.

The method may be further characterized according to, for example, the identity of the compound having inhibitory activity towards both the chemokine receptor 2 and the chemokine receptor 5. In certain embodiments, the compound has an IC₅₀ less than 0.5 μM towards chemokine receptor 2. In certain embodiments, the compound has an IC₅₀ less than 150 nM towards chemokine receptor 2. In certain embodiments, the compound has an IC₅₀ less than 0.5 μM towards chemokine receptor 5. In certain embodiments, the compound has an IC₅₀ less than 150 nM towards chemokine receptor 5. In certain embodiments, the compound has a molecular weight less than 900 g/mol.

### J. Methods for Reducing Coronavirus Entry Into a Cell Having a CCR5 Receptor

Another aspect of the invention provides methods for reducing entry of a coronavirus into a cell having a CCR5 receptor. The method comprises exposing the cell to a compound having inhibitory activity towards both the chemokine receptor 2 and the chemokine receptor 5, in order to reduce coronavirus entry into the cell.

In certain embodiments, the cell may be in a patient. Accordingly, another aspect of the invention provides methods for reducing entry of a coronavirus into a cell in a patient, wherein the cell has a CCR5 receptor. The method comprises administering to the patient in need thereof an effective amount of a compound having inhibitory activity towards both the chemokine receptor 2 and the chemokine receptor 5, in order to reduce coronavirus entry into the cell.

Another aspect of the invention provides methods for reducing entry of a coronavirus into a cell having a CCR5 receptor. The method comprises exposing the cell to a compound described herein, such as a compound of Formula I, in order to reduce coronavirus entry into the cell.

In certain embodiments, the cell may be in a patient. Accordingly, another aspect of the invention provides methods for reducing entry of a coronavirus into a cell in a patient, wherein the cell has a CCR5 receptor. The method comprises administering to the patient in need thereof an effective amount of a compound described herein, such as a compound of Formula I, in order to reduce coronavirus entry into the cell.

In certain embodiments, the compound also reduces activity of inflammatory macrophages.

The methods may be further characterized according to, for example, the identity of the compound having inhibitory activity towards both the chemokine receptor 2 and the chemokine receptor 5. In certain embodiments, the compound has an IC₅₀ less than 0.5 μM towards chemokine receptor 2. In certain embodiments, the compound has an IC₅₀ less than 150 nM towards chemokine receptor 2. In certain embodiments, the compound has an IC₅₀ less than 0.5 μM towards chemokine receptor 5. In certain embodiments, the compound has an IC₅₀ less than 150 nM towards chemokine receptor 5. In certain embodiments, the compound has a molecular weight less than 900 g/mol.

### K. General Considerations for Therapeutic Methods

General considerations that may be applied to therapeutic methods described herein (e.g., the methods described in Parts A-J above) are provided below and include, for example, the identity of the patient, the identity of the coronavirus infection, and the identity of the compound administered.

### Patients

Accordingly, the methods may be characterized according to, for example, symptoms the patient is experiencing. For example, in certain embodiments, the patient has inflammation in pulmonary tissue. In certain embodiments, the patient has mild or moderate respiratory distress. In certain embodiments, the patient has severe respiratory distress. In certain embodiments, the patient has acute respiratory distress syndrome. In certain embodiments, the patient has pneumonia. In certain embodiments, the patient presents with one or more of respiratory failure, septic shock, or multi-organ failure. In certain embodiments, the patient presents with multi-organ failure. In certain embodiments, the multi-organ failure comprises failure of a lung, liver, kidney, or heart. In certain embodiments, the patient is experiencing a hyper-immune response. In certain embodiments, the patient presents with impaired function of the brain, spinal cord, or the peripheral nervous system. In certain embodiments, the patient presents with impaired function of the brain, such as seizures or mental symptoms (e.g., a psychiatric disturbance, depression, anxiety, panic disorder, or hallucinations). In certain embodiments, the patient presents with reduced ability to smell. In certain embodiments, the patient presents with loss of ability to smell.

The methods may be characterized according to, for example, organs in the patient adversely impacted by a coronavirus infection. In certain embodiments, the coronavirus infection adversely impacts one or more of the patient's lung, liver, kidney, heart, brain, or other vital organ. In certain embodiments, the coronavirus infection adversely impacts one or more of the patient's lung, liver, kidney, heart, or brain.

The methods can be characterized according to, for example, the concentration of the pro-inflammatory cytokine in the patient. For example, in certain embodiments, the patient has a concentration of the pro-inflammatory cytokine that is greater than the average concentration in a healthy patient. In certain embodiments, the patient has a concentration of the pro-inflammatory cytokine that is at least 5%, 10%, 20%, 30%, 40%, 50%, 60%, 70%, 80%, 90% or 100% greater than the average concentration in a healthy patient. In certain embodiments, the concentration of the pro-inflammatory cytokine is measured in the blood plasma of the patient. In certain embodiments, the concentration of the pro-inflammatory cytokine is measured in a tissue sample of the patient. In certain embodiments, the concentration of the pro-inflammatory cytokine is measured in a pulmonary tissue sample of the patient.

In certain embodiments, the patient has a blood plasma concentration of one or more of IL-2, IL-7, IL-6, granulocyte-macrophage colony-stimulating factor (GM-CSF), interferon-γ inducible protein 10 (IP-10), monocyte chemoattractant protein 1 (MCP-1), RANTES, macrophage inflammatory protein 1-α (MIP-1α), or tumor necrosis factor-α (TNF-α) that is greater than the average blood plasma concentration in a healthy patient. In certain embodiments, the blood plasma concentration of one or more of IL-2, IL-7, IL-6, granulocyte-macrophage colony-stimulating factor (GM-CSF), interferon-y inducible protein 10 (IP-10), monocyte chemoattractant protein 1 (MCP-1), macrophage inflammatory protein 1-α (MIP-1α), or tumor necrosis factor-α (TNF-α) is at least 5%, 10%, 20%, 30%, 40%, 50%, 60%, 70%, 80%, 90% or 100% greater than the average blood plasma concentration in a healthy patient.

In certain embodiments, the concentration of activated macrophages in the patient is greater than the corresponding tissue average concentration of activated macrophages in a healthy patient. In certain embodiments, the concentration of activated macrophages in a tissue in the patient is at least 5%, 10%, 20%, 30%, 40%, 50%, 60%, 70%, 80%, 90% or 100% greater than the average concentration of activated macrophages in the corresponding tissue of a healthy patient. In certain embodiments, said tissue is the patient's lung, kidney, or liver.

In certain embodiments, the patient is a human. In certain embodiments, the patient is an adult human. In certain embodiments, the patient is a human over 60 years of age. In certain embodiments, the patient is a human over 65 years of age. In certain embodiments, the patient is a human over 70 years of age. In certain embodiments, the patient is a human over 75 years of age. In certain embodiments, the patient is a human over 80 years of age.

The methods can be characterized according to, for example, preexisting medical conditions of the patient. For example, in certain embodiments, the patient has compromised pulmonary function (for example, from COPD or asthma). In certain embodiments, the patient has a chronic lung disease. In certain embodiments, the patient has asthma (e.g., moderate asthma, severe asthma, or moderate to severe asthma). In certain embodiments, the patient has diabetes mellitus, is immunosuppressed (e.g., has cancer and/or is undergoing treatment for cancer). In certain embodiments, the patient has history of smoking tobacco or other substances. In certain embodiments, the patient has undergone bone marrow transplantation or organ transplantation. In certain embodiments, the patient has an immune deficiency. In certain embodiments, the patient has HIV or AIDS (e.g., poorly controlled HIV or AIDS). In certain embodiments, the patient presents with prolonged use of corticosteroids and other immune weakening medications. In certain embodiments, the patient has liver disease, chronic kidney disease, or undergoes dialysis.

In certain embodiments, the patient has diabetes. In certain embodiments, the patient has cardiovascular disease. In certain embodiments, the patient is immunocompromised. In certain embodiments, the patient has kidney or liver disease. In certain embodiments, the patient is obese.

In certain embodiments, the patient presents with impaired function of the brain, spinal cord, or the peripheral nervous system. In certain embodiments, the patient presents with impaired function of the brain, such as seizures or mental symptoms (e.g., a psychiatric disturbance, depression, anxiety, panic disorder, or hallucinations). In certain embodiments, the patient presents with reduced ability to smell. In certain embodiments, the patient presents with loss of ability to smell.

In certain embodiments, the patient has a genetic variation that produces an abnormal CCR2 that disposes the patient to increased levels and/or risk of monocyte activation and/or macrophage activation (e.g., pulmonary macrophage activation). In certain embodiments, the patient has a genetic variation that produces an abnormal CCRS that disposes the patient to increased levels and/or risk of viral infection (e.g., infection by a coronavirus, such as SARS-CoV-2.

### Coronavirus Infection

The methods can be characterized according to, for example, the identity of the coronavirus infection. For example, in certain embodiments, the coronavirus infection is an infection by SARS-CoV-2. In certain embodiments, the coronavirus infection is an infection by a coronavirus that causes severe acute respiratory syndrome (SARS) or Middle East respiratory syndrome (MERS). In certain embodiments, the coronavirus infection is an infection by a MERS coronavirus. In certain embodiments, the coronavirus infection is an infection by a SARS coronavirus.

In certain embodiments, the coronavirus infection is an infection by a variant of SARS-CoV-2. In certain embodiments, the coronavirus infection is an infection by a variant of SARS-CoV-2 having the spike protein of SARS-CoV-2. In certain embodiments, the coronavirus infection is an infection by SARS-CoV-2 or a variant thereof selected from B.1.351, Cluster 5, Lineage B.1.1.207, Lineage B.1.1.7, Variant of Concern 202102/02, Lineage B.1.1.317, Lineage B.1.1.318, Lineage B.1.351, Lineage B.1.429, Lineage B.1.525, Lineage P.1 (also known as Lineage B.1.1.28), D614G, E484K, N501Y, S477G/N, and P681H.

In certain embodiments, the coronavirus infection is an infection by SARS-CoV-2 or variant thereof having a mutation at 1, 2, 3, 4, or 5 amino acids. In certain embodiments, the coronavirus infection is an infection by SARS-CoV-2 or variant thereof having a mutation at up to 10 amino acids. In certain embodiments, the coronavirus infection is an infection by SARS-CoV-2 or variant thereof having a mutation at up to 25 amino acids.

### Result Achieved by the Method

In certain embodiments, the method may be characterized according to the result achieved. This may include, for example, the patient's reduced risk of mortality, reduced risk of post-infection morbidity, and reduced time for recovery from the infection. For example, in certain embodiments, the patient has at least a 10%, 20%, 30%, 40%, 50%, 60%, 70%, 80%, or 90% reduced risk of mortality due to the infection, relative to a patient who has not been administered the compound. In certain embodiments, the patient has at least a 10%, 20%, 30% 40%, 50%, 60%, 70%, 80%, or 90% reduced risk of post-infection morbidity due to the infection, relative to a patient who has not been administered the compound. In certain embodiments, the post-infection morbidity is reduced pulmonary function. In certain embodiments, the patient has at least a 10%, 20%, 30%, 40%, 50%, 60%, 70%, 80%, or 90% reduced time for recovery from the infection, relative to a patient who has not been administered the compound.

In certain embodiments, the method provides an increase in the amount of CD8 T-lymphocytes in the patient. In certain embodiments, the method provides restores the amount of CD8 T-lymphocytes in the patient suffering from a coronavirus infection to at least the average amount observed in a healthy patient.

### Compound Administered

The methods can be characterized according to, for example, the identity of the compound administered. As defined generally above, the compound of Formula I is represented by:

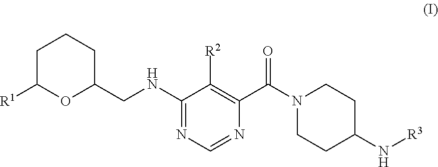

or a pharmaceutically acceptable salt thereof, wherein:

R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;

R² is C₁₋₆ alkyl or C₁₋₆ haloalkyl; and

R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I. In certain embodiments, the compound is a pharmaceutically acceptable salt of a compound of Formula I.

In certain embodiments, R¹ is phenyl substituted by 1 or 2 substituents independently selected from the group consisting of C₁₋₆ alkyl, C₁₋₆ haloalkyl, halogen, C₃₋₅ cycloalkyl, and cyano. In certain embodiments, R¹ is phenyl substituted by 1 or 2 substituents independently selected from the group consisting of C₁₋₆ alkyl and C₁₋₆ haloalkyl. In certain embodiments, R¹ is phenyl substituted by 0, 1, or 2 substituents independently selected from the group consisting of methyl, ethyl, and propyl.

In certain embodiments, R² is C₁₋₆ alkyl. In certain embodiments, R² is methyl, ethyl, or propyl.

In certain embodiments, R³ is tetrahydropyranyl substituted with 1 or 2 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl. In certain embodiments, R³ is tetrahydropyranyl that is (i) substituted with C₁₋₆ alkoxyl and (ii) optionally substituted with one C₁₋₆ alkyl, C₁₋₆ haloalkyl, halogen, or C₃₋₅ cycloalkyl. In certain embodiments, R³ is a tetrahydropyranyl that is (i) substituted with C₁₋₃ alkoxyl and (ii) optionally substituted with one C₁₋₃ alkyl, C₁₋₃ haloalkyl, halogen, or C₃₋₅ cycloalkyl. In certain embodiments, R³ is tetrahydropyranyl substituted with C₁₋₃ alkoxyl.

The description above describes multiple embodiments relating to compounds of Formula I, administered according to the therapeutic methods described herein. The patent application specifically contemplates all combinations of the embodiments.

In certain embodiments, the compound is represented by Formula I-A:

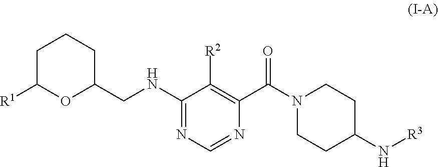

or a pharmaceutically acceptable salt thereof, wherein:

R¹ is phenyl substituted by 0, 1, or 2 substituents independently selected from the group consisting of methyl, ethyl, and propyl;

R² is methyl, ethyl, or propyl; and

R³ is a tetrahydropyranyl that is (i) substituted with C₁₋₃ alkoxyl and (ii) optionally substituted with one C₁₋₃ alkyl, C₁₋₃ haloalkyl, halogen, or C₃₋₅ cycloalkyl.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A. In certain embodiments, the compound is a pharmaceutically acceptable salt of a compound of Formula I-A.

In certain embodiments, R¹ is phenyl substituted by 1 or 2 substituents independently selected from the group consisting of methyl, ethyl, and propyl.

In certain embodiments, R³ is tetrahydropyranyl substituted with C₁₋₃ alkoxyl.

The description above describes multiple embodiments relating to compounds of Formula I-A, administered according to the therapeutic methods described herein. The patent application specifically contemplates all combinations of the embodiments.

In certain embodiments, the compound is

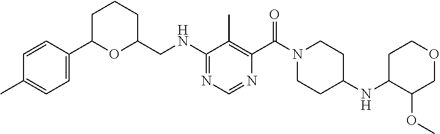

or a pharmaceutically acceptable salt thereof.

In certain embodiments, the compound is

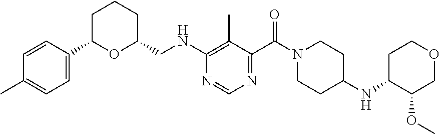

or a pharmaceutically acceptable salt thereof.

In certain embodiments, the compound is a pharmaceutically acceptable salt of:

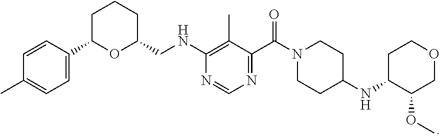

In certain embodiments, the compound is a citrate salt of:

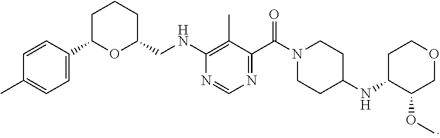

In certain embodiments, the compound is the following salt:

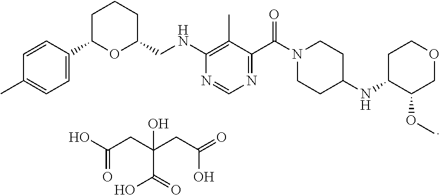

In certain embodiments, the compound is a citrate salt of:

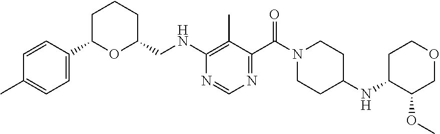

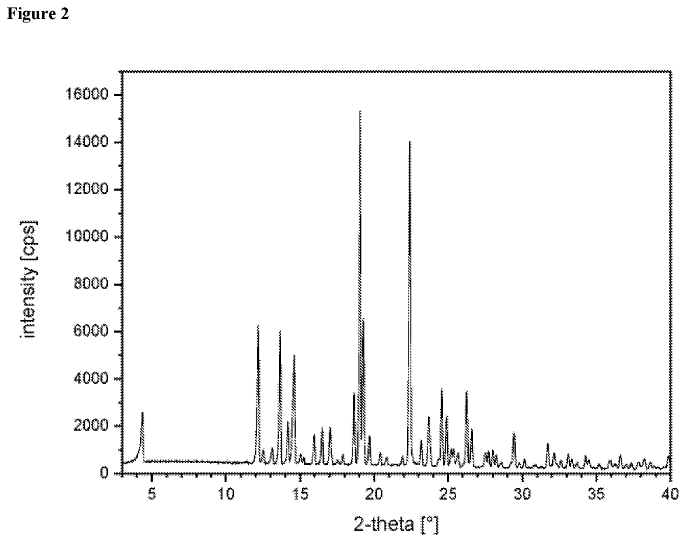

in crystalline form. In certain embodiments, the crystalline form exhibits a X-ray powder diffraction pattern comprising peaks at the following 2-theta values measured using monochromatic CuKα1 radiation of λ=1.54056 Å, 40 kV, 40 mA: 12.2±0.2, 13.7±0.2, 14.6±0.2, 19.1±0.2, and 22.4±0.2. In certain embodiments, the crystalline form exhibits a X-ray powder diffraction pattern comprising peaks at the following 2-theta values measured using monochromatic CuKα1 radiation of λ=1.54056 Å, 40 kV, 40 mA: 12.2±0.2, 13.7±0.2, 14.6±0.2, 18.7±0.2, 19.1±0.2, 22.4±0.2, 24.6±0.2, and 26.3±0.2. In certain embodiments, the relative intensity of the peak at said diffraction angles 2-theta is at least 10%. In certain embodiments, the relative intensity of the peak at said diffraction angles 2-theta is at least 15%. In certain embodiments, the crystalline form exhibits a X-ray powder diffraction pattern substantially the same as shown in FIG. 2. In certain embodiments, the crystalline form exhibits the following X-ray powder diffraction pattern expressed in terms of diffraction angle 2θ, inter-planar distances d, and relative intensity (expressed as a percentage with respect to the most intense peak), measured using monochromatic CuKα1 radiation of λ=1.54056 Å, 40 kV, 40 mA:

| 2-theta | d-value | Intensity |
|---|---|---|
| [°] | [Å] | I/I₀ [%] |
| 4.36 | 20.24 | 17 |
| 12.17 | 7.27 | 41 |
| 12.51 | 7.07 | 6 |
| 13.13 | 6.74 | 7 |
| 13.66 | 6.48 | 39 |
| 14.20 | 6.23 | 14 |
| 14.60 | 6.06 | 32 |
| 15.03 | 5.89 | 5 |
| 15.25 | 5.81 | 4 |
| 15.97 | 5.54 | 11 |
| 16.51 | 5.37 | 13 |
| 17.05 | 5.20 | 13 |
| 17.54 | 5.05 | 4 |
| 17.88 | 4.96 | 5 |
| 18.65 | 4.75 | 22 |
| 19.05 | 4.66 | 100 |
| 19.68 | 4.51 | 11 |
| 20.42 | 4.35 | 6 |
| 20.84 | 4.26 | 4 |
| 21.25 | 4.18 | 3 |
| 21.90 | 4.06 | 5 |
| 22.42 | 3.96 | 92 |
| 23.19 | 3.83 | 9 |
| 23.70 | 3.75 | 16 |
| 24.34 | 3.65 | 4 |
| 24.56 | 3.62 | 23 |
| 24.89 | 3.57 | 16 |
| 25.20 | 3.53 | 7 |
| 25.36 | 3.51 | 7 |
| 25.67 | 3.47 | 6 |
| 26.26 | 3.39 | 23 |
| 26.59 | 3.35 | 12 |
| 27.51 | 3.24 | 6 |
| 27.71 | 3.22 | 6 |
| 28.01 | 3.18 | 7 |
| 28.23 | 3.16 | 5 |
| 28.57 | 3.12 | 3 |
| 29.44 | 3.03 | 12 |
| 30.15 | 2.96 | 4 |

The citrate salt of

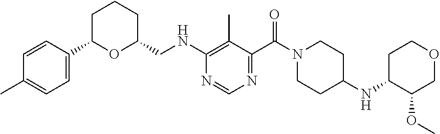

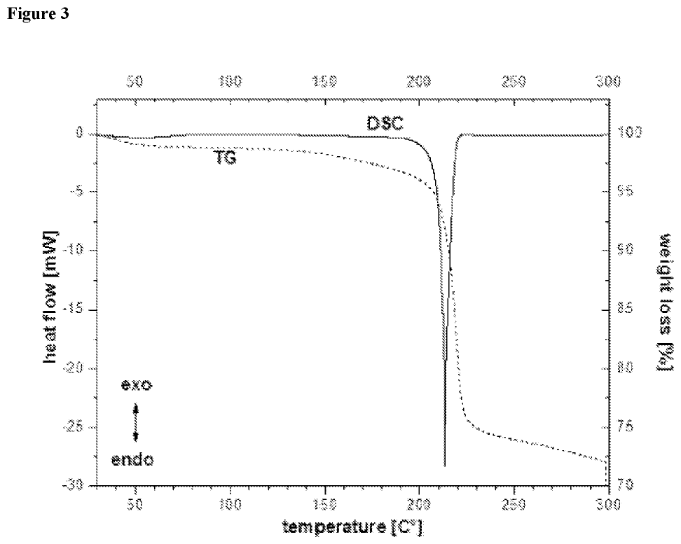

in crystalline form may be further characterized by additional features such as, for example, its Raman spectrum, melting point, and/or differential scanning calorimetry curve. In certain embodiments, the crystalline form has a Raman spectrum comprising peaks at any one or all of the following Raman shifts expressed in wavenumbers in cm⁻¹: 1718, 1242, 731, 662, 553. In certain embodiments, the crystalline form has a melting point of 212±5° C. In certain embodiments, the crystalline form has a differential scanning calorimetry curve substantially the same as shown in FIG. 3.

Additional exemplary compounds for use in the methods, pharmaceutical compositions, and medical kits described herein include those in Table I below or a pharmaceutically acceptable salt thereof.

TABLE I

| No. | Compound |
|---|---|
| I-1 | 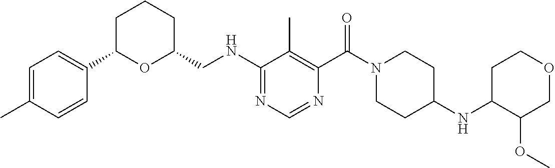 |
| I-2 | 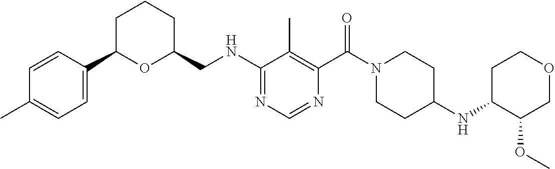 |
| I-3 | 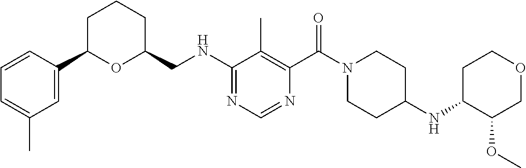 [SMILES: CO[C@H]1COCC[C@H]1NC1CCN(C(=O)c2ncnc(NC[C@@H]3CCC[C@H](c4cccc(C)c4)O3)c2C)CC1] |
| I-4 | 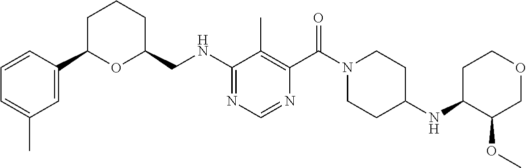 [SMILES: CO[C@@H]1COCC[C@@H]1NC1CCN(C(=O)c2ncnc(NC[C@@H]3CCC[C@H](c4cccc(C)c4)O3)c2C)CC1] |
| I-5 | 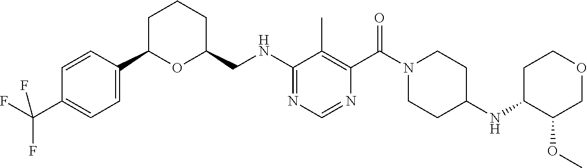 [SMILES: CO[C@H]1COCC[C@H]1NC1CCN(C(=O)c2ncnc(NC[C@@H]3CCC[C@H](c4ccc(C(F)(F)F)cc4)O3)c2C)CC1] |
| I-6 | 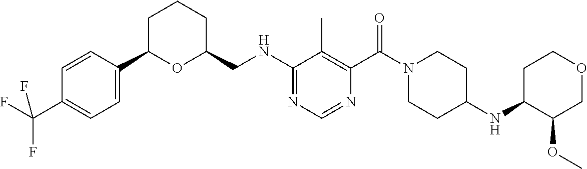 [SMILES: CO[C@@H]1COCC[C@@H]1NC1CCN(C(=O)c2ncnc(NC[C@@H]3CCC[C@H](c4ccc(C(F)(F)F)cc4)O3)c2C)CC1] |
| I-7 | 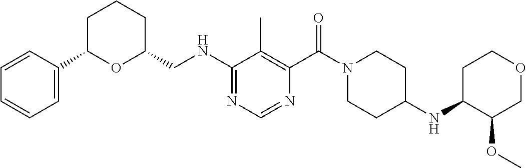 [SMILES: CO[C@@H]1COCC[C@@H]1NC1CCN(C(=O)c2ncnc(NC[C@H]3CCC[C@@H](c4ccccc4)O3)c2C)CC1] |
| I-8 | 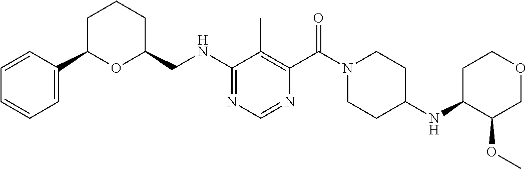 [SMILES: CO[C@@H]1COCC[C@@H]1NC1CCN(C(=O)c2ncnc(NC[C@@H]3CCC[C@H](c4ccccc4)O3)c2C)CC1] |
| I-9 | 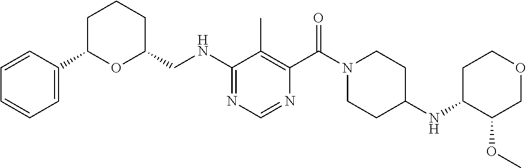 [SMILES: CO[C@H]1COCC[C@H]1NC1CCN(C(=O)c2ncnc(NC[C@H]3CCC[C@@H](c4ccccc4)O3)c2C)CC1] |
| I-10 | 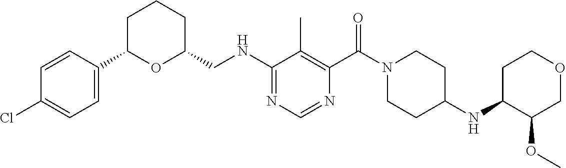 [SMILES: CO[C@@H]1COCC[C@@H]1NC1CCN(C(=O)c2ncnc(NC[C@H]3CCC[C@@H](c4ccc(Cl)cc4)O3)c2C)CC1] |
| I-11 | 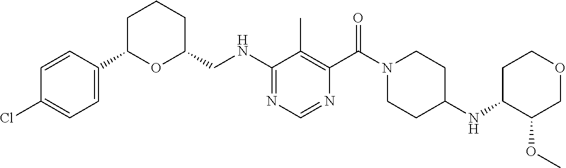 [SMILES: CO[C@H]1COCC[C@H]1NC1CCN(C(=O)c2ncnc(NC[C@H]3CCC[C@@H](c4ccc(Cl)cc4)O3)c2C)CC1] |
| I-12 | 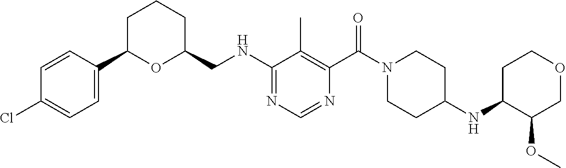 [SMILES: CO[C@@H]1COCC[C@@H]1NC1CCN(C(=O)c2ncnc(NC[C@@H]3CCC[C@H](c4ccc(Cl)cc4)O3)c2C)CC1] |
| I-13 | 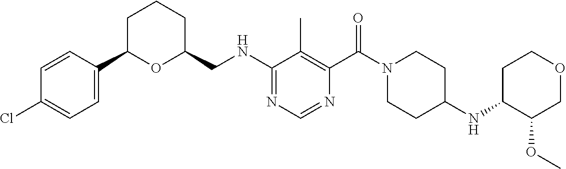 [SMILES: CO[C@H]1COCC[C@H]1NC1CCN(C(=O)c2ncnc(NC[C@@H]3CCC[C@H](c4ccc(Cl)cc4)O3)c2C)CC1] |
| I-14 | 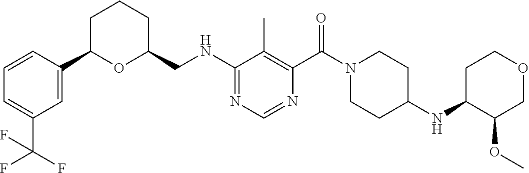 [SMILES: CO[C@@H]1COCC[C@@H]1NC1CCN(C(=O)c2ncnc(NC[C@@H]3CCC[C@H](c4cccc(C(F)(F)F)c4)O3)c2C)CC1] |
| I-15 | 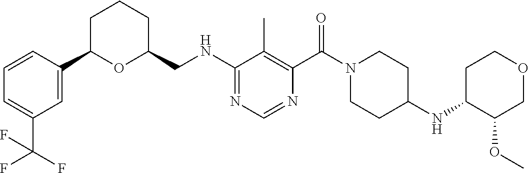 [SMILES: CO[C@H]1COCC[C@H]1NC1CCN(C(=O)c2ncnc(NC[C@@H]3CCC[C@H](c4cccc(C(F)(F)F)c4)O3)c2C)CC1] |
| I-16 | 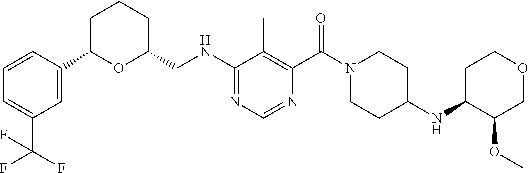 [SMILES: CO[C@@H]1COCC[C@@H]1NC1CCN(C(=O)c2ncnc(NC[C@H]3CCC[C@@H](c4cccc(C(F)(F)F)c4)O3)c2C)CC1] |
| I-17 | 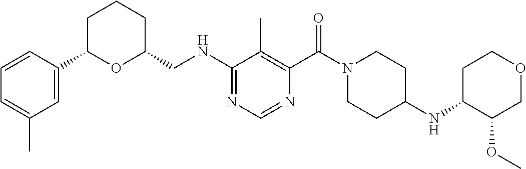 [SMILES: CO[C@H]1COCC[C@H]1NC1CCN(C(=O)c2ncnc(NC[C@H]3CCC[C@@H](c4cccc(C)c4)O3)c2C)CC1] |
| I-18 | 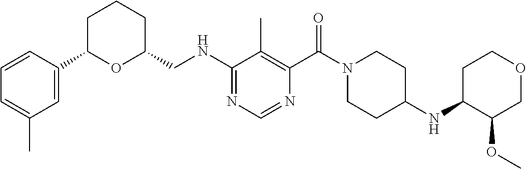 [SMILES: CO[C@@H]1COCC[C@@H]1NC1CCN(C(=O)c2ncnc(NC[C@H]3CCC[C@@H](c4cccc(C)c4)O3)c2C)CC1] |
| I-19 | 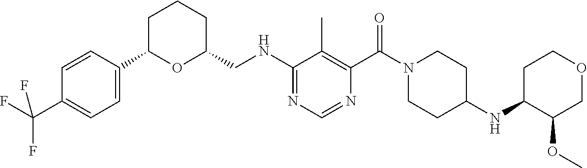 [SMILES: CO[C@@H]1COCC[C@@H]1NC1CCN(C(=O)c2ncnc(NC[C@H]3CCC[C@@H](c4ccc(C(F)(F)F)cc4)O3)c2C)CC1] |
| I-20 | 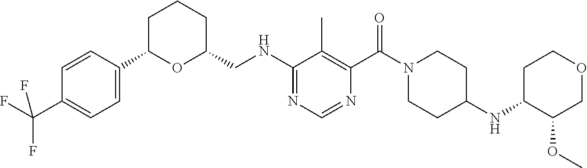 [SMILES: CO[C@H]1COCC[C@H]1NC1CCN(C(=O)c2ncnc(NC[C@H]3CCC[C@@H](c4ccc(C(F)(F)F)cc4)O3)c2C)CC1] |
| I-21 | 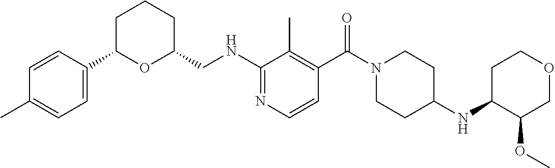 |
| I-22 | 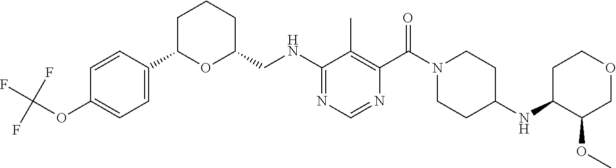 [SMILES: CO[C@@H]1COCC[C@@H]1NC1CCN(C(=O)c2ncnc(NC[C@H]3CCC[C@@H](c4ccc(OC(F)(F)F)cc4)O3)c2C)CC1] |
| I-23 | 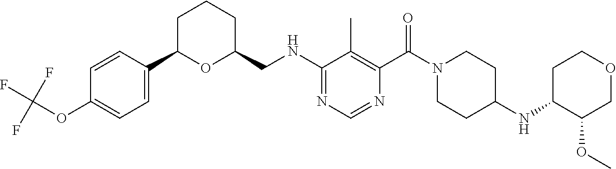 [SMILES: CO[C@H]1COCC[C@H]1NC1CCN(C(=O)c2ncnc(NC[C@@H]3CCC[C@H](c4ccc(OC(F)(F)F)cc4)O3)c2C)CC1] |
| I-24 | 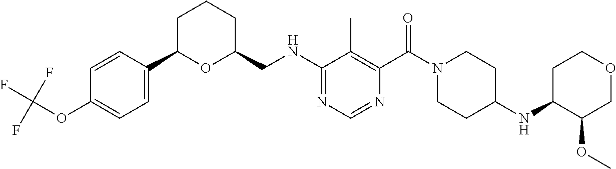 [SMILES: CO[C@@H]1COCC[C@@H]1NC1CCN(C(=O)c2ncnc(NC[C@@H]3CCC[C@H](c4ccc(OC(F)(F)F)cc4)O3)c2C)CC1] |
| I-25 | 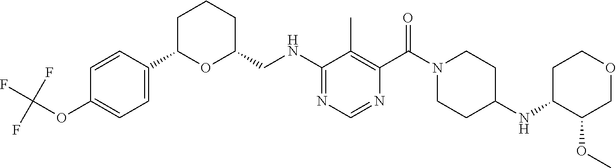 [SMILES: CO[C@H]1COCC[C@H]1NC1CCN(C(=O)c2ncnc(NC[C@H]3CCC[C@@H](c4ccc(OC(F)(F)F)cc4)O3)c2C)CC1] |
| I-26 | 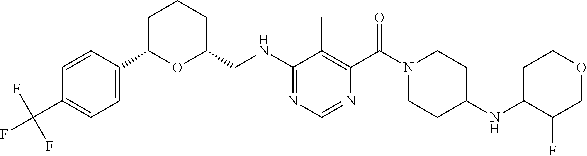 [SMILES: Cc1c(NC[C@H]2CCC[C@@H](c3ccc(C(F)(F)F)cc3)O2)ncnc1C(=O)N1CCC(NC2CCOCC2F)CC1] |
| I-27 | 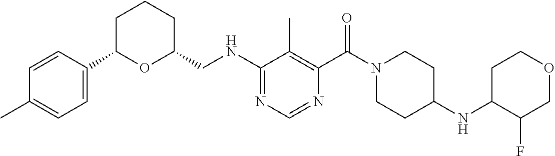 [SMILES: Cc1ccc([C@@H]2CCC[C@H](CNc3ncnc(C(=O)N4CCC(NC5CCOCC5F)CC4)c3C)O2)cc1] |
| I-28 | 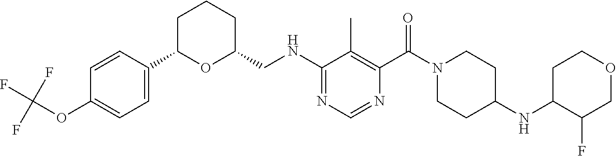 [SMILES: Cc1c(NC[C@H]2CCC[C@@H](c3ccc(OC(F)(F)F)cc3)O2)ncnc1C(=O)N1CCC(NC2CCOCC2F)CC1] |
| I-29 | 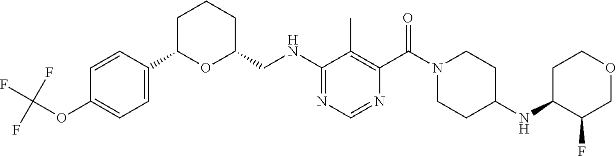 [SMILES: Cc1c(NC[C@H]2CCC[C@@H](c3ccc(OC(F)(F)F)cc3)O2)ncnc1C(=O)N1CCC(N[C@H]2CCOC[C@H]2F)CC1] |
| I-30 | 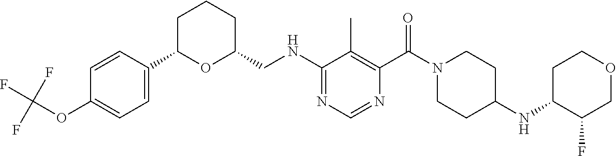 [SMILES: Cc1c(NC[C@H]2CCC[C@@H](c3ccc(OC(F)(F)F)cc3)O2)ncnc1C(=O)N1CCC(N[C@@H]2CCOC[C@@H]2F)CC1] |

The compounds may be prepared based on procedures described in, for example, WO 2011/073154 and U.S. Pat. No. 8,765,949, each of which are hereby incorporated by reference. Additional synthetic procedures and compounds are described in WO 2017/004537 and U.S. Pat. No. 10,213,428, each of which are hereby incorporated by reference

### Use of Compounds in Manufacture

Another aspect of the invention provides for the use of a compound described herein (such as a compound of Formula I or I-A, or other compounds described above) in the manufacture of a medicament. In certain embodiments, the medicament is for treating a disorder described herein, for example, for treating a coronavirus infection, reducing inflammation in a patient suffering from a coronavirus infection, reducing the impact of a pro-inflammatory cytokine in a patient suffering from a coronavirus infection, or for prophylaxis of lung injury in a patient suffering from a coronavirus infection.

### Use of Compounds in Treating Medical Disorders

Another aspect of the invention provides for the use of a compound described herein (such as a compound of Formula I or I-A, or other compounds described herein) for treating a medical disorder, such as a medical disorder described herein, for example, for treating a coronavirus infection, reducing inflammation in a patient suffering from a coronavirus infection, reducing the impact of a pro-inflammatory cytokine in a patient suffering from a coronavirus infection, or for prophylaxis of lung injury in a patient suffering from a coronavirus infection.

### II. Combination Therapy

Another aspect of the invention provides for combination therapy. Substituted (4-(amino)piperidin-1-yl)(6-(((tetrahydro-2H-pyran-2-yl)methyl)amino)pyrimidin-4-yl)methanone compounds (e.g., a compound of Formula I or I-A, or other compounds in Section I), or their pharmaceutically acceptable salts, may be used in combination with additional therapeutic agents to treat medical disorders according to the therapeutic methods described in Section I.

Exemplary additional therapeutic agents that may be used as part of a combination therapy in treating a coronavirus infection and related medical conditions, include, for example, an anti-viral agents and anti-inflammatory agents. In certain embodiments, the additional therapeutic agent is an anti-viral agent, which may be, for example, immune globulins from patent that has recovered from a coronavirus infection (e.g., SARS-CoV-2). In certain embodiments, the additional therapeutic agent is a monoclonal antibody against SARS-CoV-2. In certain embodiments, the additional therapeutic agent is an anti-inflammatory agent, such as an agent that inhibits the activity of one or more of IL-1, IL-6, and tumor necrosis factor (TNF). In certain embodiments, the additional therapeutic agent is non-steroidal anti-inflammatory drug, corticosteroid, interferon, or agent that reduces suppression of interferon.

In certain embodiments, the additional therapeutic agent used as part of a combination therapy is favipiravir, remdesivir, EIDD-2801 (remdesivir isobutyrate), lopinavir, ritonavir, tocilizumab, sarilumab, losartan, melatonin, mercaptopurine, sirolimus (with or without dactinomycin), toremifene, emodin, chloroquine, or a pharmaceutically acceptable salt thereof.

In certain embodiments, the additional therapeutic agent is antibiotic agent or anti-fungal agent. Antibiotic agents may be particular beneficial in treating patients suffering from a secondary infection, such as pneumonia. For patients that a genetic predisposition to a coronavirus infection (e.g., SARS-CoV-2), the additional therapeutic agent may be an agent that reduces impact of the patient's predisposition to a coronavirus infection. In certain embodiments, such additional therapeutic agent may be, for example, an iRNA, oligonucleotide, cell based therapy (e.g., a pluripotent cell, a mesenchymal stem cell, or a placental derived cell), or gene therapy.

The doses and dosage regimen of the active ingredients used in the combination therapy may be determined by an attending clinician. In certain embodiments, the substituted (4-(amino)piperidin-1-yl)(6-(((tetrahydro-2H-pyran-2-yl)methyl)amino)pyrimidin-4-yl)methanone compounds (e.g., a compound of Formula I or I-A, or other compounds in Section I) and the additional therapeutic agent(s) are administered in doses commonly employed when such agents are used as monotherapy for treating the disorder. In other embodiments, the substituted (4-(amino)piperidin-1-yl)(6-(((tetrahydro-2H-pyran-2-yl)methyl)amino)pyrimidin-4-yl)methanone compounds (e.g., a compound of Formula I or I-A, or other compounds in Section I) and the additional therapeutic agent(s) are administered in doses lower than the doses commonly employed when such agents are used as monotherapy for treating the disorder. In certain embodiments, the substituted (4-(amino)piperidin-1-yl)(6-(((tetrahydro-2H-pyran-2-yl)methyl)amino)pyrimidin-4-yl)methanone compounds (e.g., a compound of Formula I or I-A, or other compounds in Section I) and the additional therapeutic agent(s) are present in the same composition, which is suitable for oral administration.

In certain embodiments, the substituted (4-(amino)piperidin-1-yl)(6-(((tetrahydro-2H-pyran-2-yl)methyl)amino)pyrimidin-4-yl)methanone compounds (e.g., a compound of Formula I or I-A, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

### III. Pharmaceutical Compositions

As indicated above, the invention provides pharmaceutical compositions, which comprise one or more of the compounds described above, formulated together with one or more pharmaceutically acceptable carriers. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a substituted (4-(amino)piperidin-1-yl)(6-(((tetrahydro-2H-pyran-2-yl)methyl)amino)pyrimidin-4-yl)methanone compound described herein in a therapeutically effective amount for the treatment of a medical disorder described herein.

### IV. Medical Kits

Another aspect of the invention provides a medical kit comprising, for example, (i) a compound described herein, such as a substituted (4-(amino)piperidin-1-yl)(6-(((tetrahydro-2H-pyran-2-yl)methyl)amino)pyrimidin-4-yl)methanone compound described herein, which may be, for example, a compound of Formula I or I-A, and (ii) instructions for administering the compound according to methods described herein. In certain embodiments, the medical kit comprises (i) a pharmaceutical composition comprising a substituted (4-(amino)piperidin-1-yl)(6-(((tetrahydro-2H-pyran-2-yl)methyl)amino)pyrimidin-4-yl)methanone compound described herein, such as a compound of Formula I or I-A, and (ii) instructions for administering the pharmaceutical composition according to methods described herein.

### EXAMPLES

The invention now being generally described, will be more readily understood by reference to the following examples, which are included merely for purposes of illustrating certain aspects and embodiments of the present invention, and are not intended to limit the invention.

### Example 1

### Preparation of (4-((3R,4R)-3-Methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate (1)

The title compound was prepared and characterized according to the experimental procedures and results are provided below. The title compound, Compound 1, has the following formula:

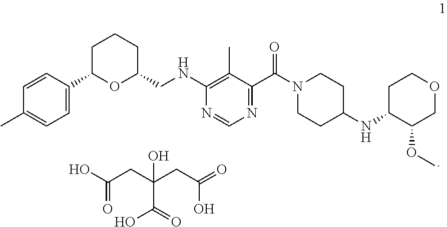

### Part I: Description of Analytical Methods Used

Provided below is a description of analytical methods used to characterize compound 1.

ESI Mass Spectrometry (ESI+)

| Instrument | QTOF 2 (Micromass, Manchester, UK) |
|---|---|
| Instrument control software | Masslynx 4.1 |
| Ion source | ESI + (Lockspray source) |
| Lockspray/DXC | on/off |
| Calibration | 0.1% Phosphoric acid in |
| | acetonitrile/water (1:1), |
| | lockmass calibration |
| Resolution MS1(LM/HM) | 5/5 |
| Resolving power (FWHM) | 16000 at m/z 491 (W mode) |
| MCP voltage | 2200 V |
| Capillary voltage | +2.8 kV |
| Cone voltage | 25 V |
| Collision energy | 5 V |
| Collision gas | Argon |
| Source temperature | 120° C. |
| Desolvation temperature | 150° C. |
| Cone gas | nitrogen 75 L/h |
| Desolvation gas | nitrogen 450 L/h |
| Spray solvent | acetonitrile/water 9:1 |
| Syringe pump | Harvard Apparatus 55-2222 |
| Spray solvent flow rate | 5 μL/min |
| Sample concentration | 5 ng/μL spray solvent |
| Reagents | acetonitrile (ULC/MS, Biosolve) |
| | water (purified by Milli-Q-system) |
| Scan range | 50-1000 u (TOF scan, profile data) |
| Scan time | 2.9 s |
| No. of scans combined | 20 |
| Accurate mass | Center 5 points/80%, Np = 0.35, |
| determination | lockmass: 588.8692 |
| Data threshold | 1.0% |

¹H NMR Spectroscopy

| | |
|---|---|
| Instrument | Bruker DRX 400 |
| Frequency | 400.13 MHz |
| Software | TopSpin ® version 1.3 PL8 |
| Pulse program | zg30 |
| Solvent | DMSO-d₆ |
| Concentration | 10.3 mg/0.6 mL |
| Temperature | 30° C. |
| Calibration | TMS (δ = 0.00 ppm) |
| Sweep width | 8013 Hz |
| Size | 64K data points |
| Pulse width | 30 degree |
| Relaxation delay | 10 s |
| Number of scans | 32 |
| Dummy scans | 8 |
| Apodization | zerofilling to 128K data points |
| | Gaussian multiplication (GB: 0.25, LB: −0.25 Hz) |

¹³C NMR Spectroscopy

| | |
|---|---|
| Instrument | Bruker DRX 400 |
| Frequency | 100.61 MHz |
| Software | TopSpin ® version 1.3 PL8 |
| Pulse program | Zgpg |
| Solvent | DMSO-d₆ |
| Concentration | 10.3 mg/0.6 ml |
| Temperature | 30° C. |
| Calibration | DMSO-d₆ (δ = 39.5 ppm) |
| Sweep width | 27778 Hz |
| Size | 64K data points |
| Pulse width | 90 degree |
| Relaxation delay | 4 s |
| Number of scans | 4096 |
| Dummy scans | 32 |
| Apodization | zerofilling to 128K data points |
| | Exponential multiplication (LB: 2.5 Hz) |

### X-ray Powder (XRPD) Diagram

X-ray powder diagrams were generated using a STOE-STADI P-diffractometer in transmission mode fitted with a MYTHEN-detector and a Cu-anode as X-ray source with monochromatic CuKα1 radiation (λ=1.54056 Å, 40 kV, 40 mA).

### FT-RAMAN Spectroscopy

Samples have been measured in boiling point tubes using a Bruker RAM II FT-Raman Module instrument, resolution 2 cm⁻¹, 64 scans, laser power 500 mW (focused laser). Analysis: scaling of vector in spectral range 3500 cm⁻¹-50 cm⁻¹.

### Differential Scanning Calorimetry—Melting Point

The compounds are characterized by a melting point determined by Differential Scanning Calorimetry (DSC), evaluated by the peak maximum or onset temperature. The heating rate of the experiment is 10° C./min. The values given were determined using a DSC instrument from the Q-series™ of TA Instruments.

### ThermoGravimetry (TG)

Thermal gravimetry data were collected with a TG instrument from the Q-series of TA Instruments. This method measures weight changes in a material as a function of temperature under a controlled atmosphere.

### Dynamic Vapour Sorption (DVS)

Sorption isotherms were generated using an IGAsorp water sorption monitor from Hiden Isochema. Adsorption and desorption isotherms were obtained at 25° C. with 10% r.h. step intervals ranging from 10% r.h. to 90% r.h.

For BR salt form only: Sorption isotherms were registered on a DVS-1 water sorption monitor from Surface Measurement Systems.

### Solubility

Solubility was determined using an automated shake flask method (at room temperature) and quantitation of the dissolved drug substance was determined by UV-spectroscopy within this automated setup.

### Part II: Preparation of (4-((3R,4R)-3-Methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate (1)

Exemplary procedures for making the title compound are provided below, along with physical characterization data. The preparation procedures include two different routes for making the title compound.

### Preparation Option a) Preparation of Citrate Salt Starting From Free Base I:

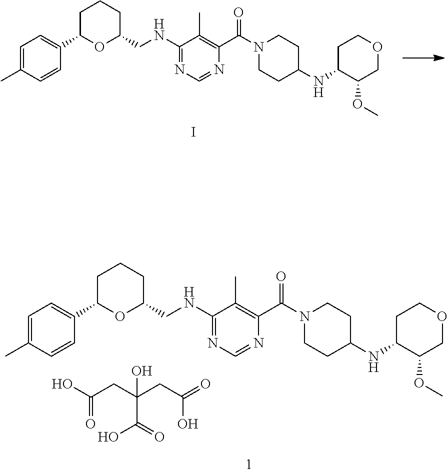

To a solution of the free base I (200 mg, 0.372 mmol) in ethyl acetate (2 mL) is added citric acid mono hydrate (78.2 mg; 0.372 mmol). The solution is stirred overnight (18 h). The suspension is filtered and the product is dried at 40° C. in vacuo to yield 140 mg 0.192 mmol (52%) colorless crystals. Physical characterization data for citrate salt 1 is provided below.

NMR (¹H, 400 MHz, DMSO-d₆): 11.7-8.5 (2H, broad), 8.34 (1H, s), 7.22 (2H, m), 7.12 (2H, m), 7.08 (1H, t), 4.49 (1H, m), 4.31 (1H, d), 4.09 (1H, m), 3.85 (1H, m), 3.74 (1H, m), 3.57-3.44 (2H, m), 3.48 (1H, m), 3.47 (1H, m), 3.35 (3H, s), 3.35 (1H, m), 3.33 (1H, m), 3.29 (1H, m), 3.27 (1H, m), 3.04 (1H, m), 2.84 (1H, m), 2.58 (2H, d), 2.50 (2H, d), 2.28 (3H, s), 2.12 (1H, m), 1.94 (1H, m), 1.91 (3H, s), 1.88 (1H, m), 1.78 (1H, m), 1.76 (1H, m), 1.70 (1H, m), 1.66 (1H, m), 1.63 (1H, m), 1.40 (1H, m), 1.40 (1H, m), 1.37 (1H, m), 1.24 (1H, m) (includes rotamers).

NMR (¹³C, 100 MHz, DMSO-d₆): 176.6, 171, 165.4, 161.0, 156.6, 155.4, 140.3, 136.0, 128.5, 125.6, 109.3, 78.5, 75.4, 72.4, 72.2, 71.2, 64.8, 64.4, 64.4, 55.5, 55.5, 51.5, 51.4, 50.2, 45.6, 44.1, 44.1, 38.8, 33.3, 29.6, 28.7, 28.7, 25.1, 23.1, 20.6, 11.7 (includes rotamers).

HRMS (ESI): m/z 538.3400 ([M+H]⁺; C₃₀H₄₄N₅O₄).

FT-RAMAN spectrum (characteristic bands) [cm⁻¹]: 1718, 1242, 731, 662, 553.

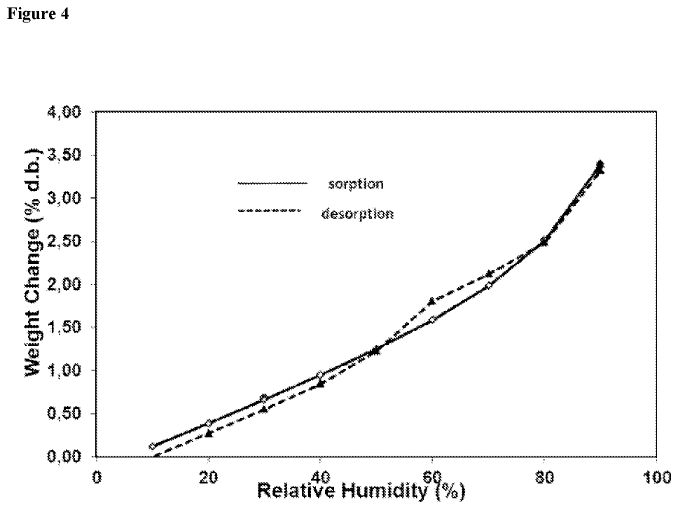

See Table II below and FIGS. 2-4 for additional characterization data.

### Preparation Option b) Amide Coupling Followed by Preparation of Citrate Salt:

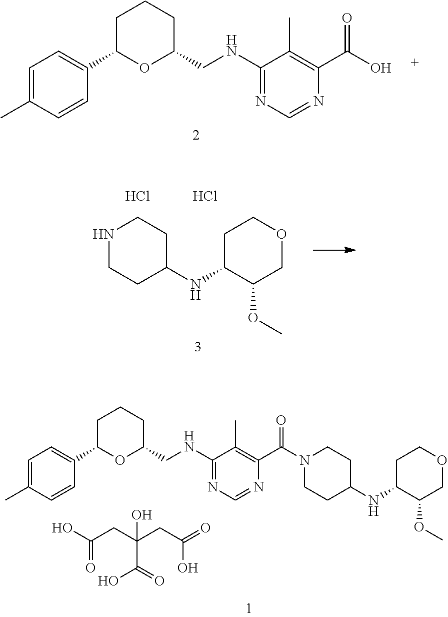

4.99 kg (30.75 mol) of 1,1′-carbonyldiimidazole are added to a suspension of 10.0 kg (29.29 mol) of 2 in 75 L of 2-methyltetrahydrofuran at 50° C. The powder funnel is rinsed with 5 L 2-methyltetrahydrofuran. The reaction mixture is stirred for 70 min at 50° C. Then, 8.83 kg (30.75 mol) of 3 are added to the reaction mixture and the funnel is rinsed with 5 L 2-methyltetrahydrofuran. Next, 7.41 kg (73.23 mol) of triethylamine and 10 L of 2-methyltetrahydrofuran are added and the reaction mixture is stirred for 1 h under reflux. Then, the mixture is cooled to 60° C. and a solution of 6.07 kg (43.94 mol) of potassium carbonate in 55 L water is added and the phases are separated at 55° C. The organic layer is washed with 60 L water and 80 L of solvent are removed by distillation in vacuo. The resulting residue is diluted with 80 L of isopropyl alcohol and 55 L of solvent is removed by distillation in vacuo. The resulting residue is diluted with 40 L of isopropyl alcohol and 40 L of solvent is removed by distillation in vacuo. Next, 5.85 kg (27.83 mol) of citric acid monohydrate in 11 L of water are added and the dropping funnel is rinsed with 30 L of isopropyl alcohol. The reaction mixture is heated to 75° C., stirred until a solution is formed, and then filtrated. The filter is rinsed with a mixture of 2 L of water and 20 L of isopropyl alcohol. Then, the filtrate is diluted with 30 L of isopropyl alcohol and seeded with 100 g of 1 as obtained in option a) at 65° C. Next, the mixture is cooled to 55° C. within 30 minutes and then further stirred for 1 h at 55° C. The resulting suspension is diluted with 60 L of isopropyl alcohol within 1 h at 55° C. and then cooled to 20° C. within 3 h. Then, the suspension is stirred for 17 h at 20° C. and isolated by filtration. The filter cake is washed twice with a mixture of 19 L of isopropyl alcohol and 1 L of water, each. The product is dried at 50° C. in vacuo to yield 17.76 kg of compound (83%). Physical characterization data for compound 1 is provided below.

NMR (¹H, 400 MHz, DMSO-d₆): 11.7-8.5 (2H, broad), 8.34 (1H, s), 7.22 (2H, m), 7.12 (2H, m), 7.08 (1H, t), 4.49 (1H, m), 4.31 (1H, d), 4.09 (1H, m), 3.85 (1H, m), 3.74 (1H, m), 3.57-3.44 (2H, m), 3.48 (1H, m), 3.47 (1H, m), 3.35 (3H, s), 3.35 (1H, m), 3.33 (1H, m), 3.29 (1H, m), 3.27 (1H, m), 3.04 (1H, m), 2.84 (1H, m), 2.58 (2H, d), 2.50 (2H, d), 2.28 (3H, s), 2.12 (1H, m), 1.94 (1H, m), 1.91 (3H, s), 1.88 (1H, m), 1.78 (1H, m), 1.76 (1H, m), 1.70 (1H, m), 1.66 (1H, m), 1.63 (1H, m), 1.40 (1H, m), 1.40 (1H, m), 1.37 (1H, m), 1.24 (1H, m) (includes rotamers).

NMR (¹³C, 100 MHz, DMSO-d₆): 176.6, 171, 165.4, 161.0, 156.6, 155.4, 140.3, 136.0, 128.5, 125.6, 109.3, 78.5, 75.4, 72.4, 72.2, 71.2, 64.8, 64.4, 64.4, 55.5, 55.5, 51.5, 51.4, 50.2, 45.6, 44.1, 44.1, 38.8, 33.3, 29.6, 28.7, 28.7, 25.1, 23.1, 20.6, 11.7 (includes rotamers).

HRMS (ESI): m/z 538.3400 ([M+H]⁺; C₃₀H₄₄N₅O₄).

FT-RAMAN spectrum (characteristic bands) [cm⁻¹]: 1718, 1242, 731, 662, 553.

See Table II below and FIGS. 2-4 for additional characterization data.

### Solid State Properties of Compound 1

Various solid state properties of compound 1 are described below.

### Appearance

In the solid state, (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate is a white microcrystalline material.

### Sorption Behavior

(4-((3R,4R)-3-Methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate shows stability against relative humidity up to 80%. An uptake of 2.6% water is observed. The water uptake is reversible, and after the sorption experiment the compound still remains as solid material. All other salts turned into liquid phase at higher relative humidity (depending on the salt form starting at 60-70% relative humidity).

### Crystallinity and Polymorphism

### Compound 1

Compound 1 is highly crystalline as can be seen in the X-ray powder diffraction diagram in FIG. 2. The X-ray powder reflection and intensities (standardized) are shown in Table II.

TABLE II

| 2-theta | d-value | Intensity |
|---|---|---|
| [°] | [Å] | I/I₀ [%] |
| 4.36 | 20.24 | 17 |
| 12.17 | 7.27 | 41 |
| 12.51 | 7.07 | 6 |
| 13.13 | 6.74 | 7 |
| 13.66 | 6.48 | 39 |
| 14.20 | 6.23 | 14 |
| 14.60 | 6.06 | 32 |
| 15.03 | 5.89 | 5 |
| 15.25 | 5.81 | 4 |
| 15.97 | 5.54 | 11 |
| 16.51 | 5.37 | 13 |
| 17.05 | 5.20 | 13 |
| 17.54 | 5.05 | 4 |
| 17.88 | 4.96 | 5 |
| 18.65 | 4.75 | 22 |
| 19.05 | 4.66 | 100 |
| 19.68 | 4.51 | 11 |
| 20.42 | 4.35 | 6 |
| 20.84 | 4.26 | 4 |
| 21.25 | 4.18 | 3 |
| 21.90 | 4.06 | 5 |
| 22.42 | 3.96 | 92 |
| 23.19 | 3.83 | 9 |
| 23.70 | 3.75 | 16 |
| 24.34 | 3.65 | 4 |
| 24.56 | 3.62 | 23 |
| 24.89 | 3.57 | 16 |
| 25.20 | 3.53 | 7 |
| 25.36 | 3.51 | 7 |
| 25.67 | 3.47 | 6 |
| 26.26 | 3.39 | 23 |
| 26.59 | 3.35 | 12 |
| 27.51 | 3.24 | 6 |
| 27.71 | 3.22 | 6 |
| 28.01 | 3.18 | 7 |
| 28.23 | 3.16 | 5 |
| 28.57 | 3.12 | 3 |
| 29.44 | 3.03 | 12 |
| 30.15 | 2.96 | 4 |

In Table II above, the value “2-theta [°]” denotes the angle of diffraction in degrees and the d-value [Å] denotes the specified distances in Å between the lattice planes.

The crystalline citrate salt of (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone is characterized in that the x-ray powder diagram has, inter alia, the characteristic values 2-theta=19.1° (100% relative intensity), 22.4° (92% relative intensity), 12.2° (41% relative intensity), 13.7° (39% relative intensity), and 14.6° (32% relative intensity) (which are the most prominent peaks in the diagram of FIG. 2, Table II).

### Example 2

### CCR2 and CCR5 Inhibitory Activity of (4-((3R,4R)-3-Methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate (1)

The CCR2 and CCR5 inhibitory activity of (4-((3R,4R)-3-methoxytetrahydro-pyran-4-ylamino)piperidin-1-yl)(5-methyl-6-(((2R,6S)-6-(p-tolyl)tetrahydro-2H-pyran-2-yl)methylamino)pyrimidin-4-yl)methanone citrate was evaluated against human CCR2 receptors and human CCR5 receptors. Experimental results are provided in the table below.

TABLE III
CCR2 and CCR5 Inhibitory Activity

| Receptor | Kᵢ (nM) | IC₅₀ (nM) |
|---|---|---|
| CCR2 | 4.2 | 50 |
| CCR5 | 75 | 88 |

### INCORPORATION BY REFERENCE

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a coronavirus infection, comprising administering to a patient in need thereof a therapeutically effective amount of a compound of Formula I in order to treat the coronavirus infection, wherein Formula I is represented by: 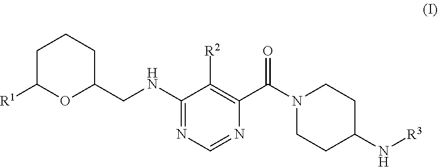
or a pharmaceutically acceptable salt thereof, wherein:
R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;
R² is C₁₋₆ alkyl or C₁₋₆ haloalkyl; and
R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

2. The method of claim 1, wherein the patient presents with inflammation due to the coronavirus infection.

3. A method of reducing inflammation in a patient suffering from a coronavirus infection, comprising administering to a patient in need thereof an effective amount of a compound of Formula I in order to reduce inflammation, wherein Formula I is represented by: 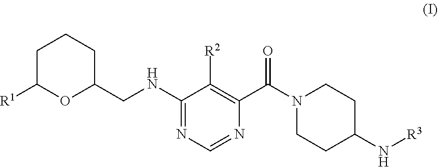
or a pharmaceutically acceptable salt thereof, wherein:
R¹ is phenyl or a 5-6 membered heteroaryl, each of which is substituted by 0, 1, 2, or 3 substituents independently selected from the group consisting of C₁₋₆ alkyl, C₁₋₆ haloalkyl, halogen, C₁₋₆ alkoxyl, C₁₋₆ haloalkoxyl, C₃₋₅ cycloalkyl, and cyano;
R² is C₁₋₆ alkyl or C₁₋₆ haloalkyl; and
R³ is a 4-8 membered heterocyclyl substituted with 0, 1, 2, or 3 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

4-9. (canceled)

10. The method of claim 1, wherein the patient has inflammation in pulmonary tissue.

11. The method of claim 1, wherein the patient has mild or moderate respiratory distress.

12. The method of claim 1, wherein the patient has severe respiratory distress.

13. (canceled)

14. (canceled)

15. The method of claim 1, wherein the patient is experiencing a hyper-immune response.

16. The method of claim 1, wherein the coronavirus infection is an infection by SARS-CoV-2.

17. The method of claim 1, wherein the patient is an adult human.

18. The method of claim 1, wherein R¹ is phenyl substituted by 1 or 2 substituents independently selected from the group consisting of C₁₋₆ alkyl, C₁₋₆ haloalkyl, halogen, C₃₋₅ cycloalkyl, and cyano.

19. (canceled)

20. The method of claim 1, wherein R³ is a 4-8 membered heterocycloalkyl substituted with 1 or 2 substituents independently selected from the group consisting of C₁₋₆ alkoxyl, C₁₋₆ haloalkoxyl, C₁₋₆ alkyl, C₁₋₆ haloalkyl, halogen, hydroxyl, cyano, and C₃₋₅ cycloalkyl.

21. (canceled)

22. The method of claim 18, wherein R³ is tetrahydropyranyl that is (i) substituted with C₁₋₆ alkoxyl and (ii) optionally substituted with one C₁₋₆ alkyl, C₁₋₆ haloalkyl, halogen, or C₃₋₅ cycloalkyl.

23. (canceled)

24. The method of claim 1, wherein the compound is represented by Formula I-A: 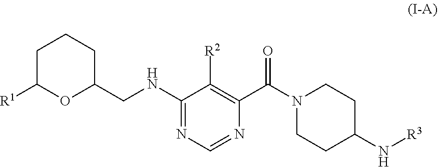
or a pharmaceutically acceptable salt thereof, wherein:
R¹ is phenyl substituted by 0, 1, or 2 substituents independently selected from the group consisting of methyl, ethyl, and propyl;
R² is methyl, ethyl, or propyl; and
R³ is a tetrahydropyranyl that is (i) substituted with C₁₋₃ alkoxyl and (ii) optionally substituted with one C₁₋₃ alkyl, C₁₋₃ haloalkyl, halogen, or C₃₋₅ cycloalkyl.

25. The method of claim 1, wherein the compound is 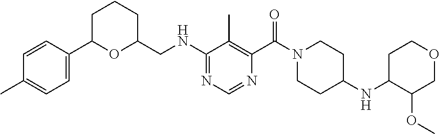
or a pharmaceutically acceptable salt thereof.

26. The method of claim 1, wherein the compound is 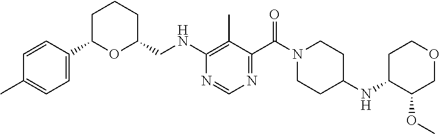
or a pharmaceutically acceptable salt thereof.

27. The method of claim 1, wherein the compound is a pharmaceutically acceptable salt of:

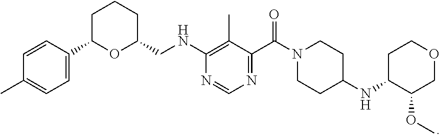

28. The method of claim 1, wherein the compound is a citrate salt of:

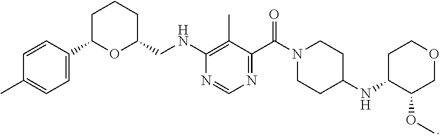

29-38. (canceled)

39. A method of treating a coronavirus infection, comprising administering to a patient in need thereof a therapeutically effective amount of a compound having inhibitory activity towards both the chemokine receptor 2 and the chemokine receptor 5, in order to treat the coronavirus infection.

40. (canceled)

41. The method of claim 39, wherein the compound has an IC₅₀ less than 150 nM towards chemokine receptor 2.

42. (canceled)

43. The method of claim 41, wherein the compound has an IC₅₀ less than 150 nM towards chemokine receptor 5.

44-51. (canceled)

52. The method of claim 39, wherein the coronavirus infection is an infection by SARS-CoV-2.

53-55. (canceled)
